(19) United States
(12) Patent Application Publication
(10) Pub. No.: US 2026/0325083 A1
(43) Pub. Date: Oct. 22, 2026

(54) LENS-TYPE MEDICAL DETECTION DEVICE

(71) Applicant: CHI-HSIU CHEN, New Taipei City (TW)

(72) Inventor: CHI-HSIU CHEN, New Taipei City (TW)

(21) Appl. No.: 19/185,289

(22) Filed: Apr. 22, 2025

(51) Int. Cl. A61B 5/00; A61B 5/0205; A61B 5/11

(52) U.S. Cl. CPC A61B 5/7465; A61B 5/0022; A61B 5/02055; A61B 5/1117; A61B 5/7264; A61B 5/7405

(57) ABSTRACT

A lens-type medical detection device includes a SoC connected to a display, a communication module, a remote measurement module, and a fall detection module. The communication module establishes connection with a cloud server and a contact person. The cloud server includes an artificial intelligence model. A light-emitting unit illuminates a subject and a lens successively captures multiple images, each of which is transmitted to the cloud server to be subjected to computation performed by the artificial intelligence model to generate physiological information, which is transmitted back and outputted to the display. The physiological information includes heartbeat, heart rate, blood pressure, blood oxygen, blood sugar, and lactic acid. Based on the physiological information, the artificial intelligence model generates a comprehensive health assessment for prompting proper handling to the subject and the family. The fall detection module detects a fall of the subject and notifies a contact person through the communication module.

### BACKGROUND OF THE INVENTION

### Technical Field of the Invention

The present invention relates generally to a physiological detection device, and more particularly to a lens-type medical detection device that uses a lens, infrared light, and artificial intelligence (AI) algorithms to detect physiological information, such as heartbeat, blood sugar, blood oxygen, blood pressure, lactic acid, and alcohol of the human body in a non-contact manner.

### Description of the Prior Art

The evolution of non-contact physiological signal measurement technology is based on photoplethysmography (PPG), starting from emitting light of a specific wavelength to measure blood pressure, heart rate, blood oxygen, and heart rate variability in a close-range/contact manner, and then developing into remote photoplethysmography (rPPG) technology.

Although in the prior art, contact physiological signal measurement technology has been widely used in the measurement/estimation of physiological signals, such as blood pressure, heart rate, blood oxygen, and heart rate variability, non-contact physiological signal measurement technology still has a lot of application problems to be improved. In the prior art, the measurement/estimation model used is just a general calculation formula, particularly the model/formula used to estimate blood pressure, which does not take into account the physiological differences between different users/subjects. Therefore, the accuracy of blood pressure measurement and estimation still cannot achieve the ideal effect. In view of this, the prior art physiological signal measurement device still needs to be improved.

### SUMMARY OF THE INVENTION

In view of the above-discussed shortcomings, the present invention provides a lens-type medical detection device, which comprises a system on a chip (SoC); a display, which is electrically connected to the SoC; a communication module, which is electrically connected to the SoC, wherein the communication module establishes connection with a cloud server and at least one contact person, the cloud server comprising an artificial intelligence model; at least one remote measurement module, which is electrically connected to the SoC and comprises at least one light-emitting unit and at least one lens, the light-emitting unit comprising at least one infrared light-emitting diode and at least one visible light emitting element, wherein the light-emitting unit is operable to illuminate a subject and the lens is operable to successively capture a plurality of images and each of the images is transmitted to the cloud server to be subjected to computation performed by the artificial intelligence model to generate physiological information, the physiological information being transmitted back and outputted to the display, wherein the physiological information includes heartbeat, heart rate, blood pressure, blood oxygen, blood sugar, and lactic acid, and the artificial intelligence model generates a comprehensive health assessment according to the physiological information, so that the comprehensive health assessment reminds the subject and family thereof to carry out corresponding handling; and a fall detection module, which is electrically connected to the SoC, wherein the fall detection module receives a behavior trajectory in real time in order to carry out fall judgement using an algorithm of the SoC and to notify the contact person through the communication module.

Preferably, the remote measurement module further comprises a complementary metal oxide semiconductor.

Preferably, the SoC is further connected to a body temperature detector, and the body temperature detector provides detection of a body temperature of the subject for integration into the physiological information.

Preferably, the SoC is further connected to a storage module, and the storage module includes an address book for storing information of the contact person.

Preferably, the SoC is connected to at least one speed dial key, and the speed dial key is connected with the information of the at least one contact person in the address book.

Preferably, the SoC is connected to at least one emergency key and a loud speaker, and triggering of the emergency key makes the loud speaker emit a warning sound effect.

Preferably, the storage module includes a calendar therein for recording and remining the subject to take medicine or go to the hospital for consultation.

Preferably, the artificial intelligence model uses remote photoplethysmography (rPPG) and face recognition technology to process each of the images, in order to obtain the physiological information.

Preferably, each of the images includes the face, chest, arms and wrists of the subject.

### BRIEF DESCRIPTION OF THE DRAWINGS

FIG. 1 is a schematic diagram showing an overall structure of the present invention.

FIG. 2 is a schematic diagram showing a remote measurement module of the present invention.

FIG. 3 is a schematic diagram showing an artificial intelligence model processing an image according to the present invention.

FIG. 4 is a flow chart illustrating fall judgement according to the present invention.

FIG. 5 is a schematic diagram showing an outside appearance of a lens-type medical detection device according to the present invention.

### DETAILED DESCRIPTION OF THE PREFERRED EMBODIMENTS

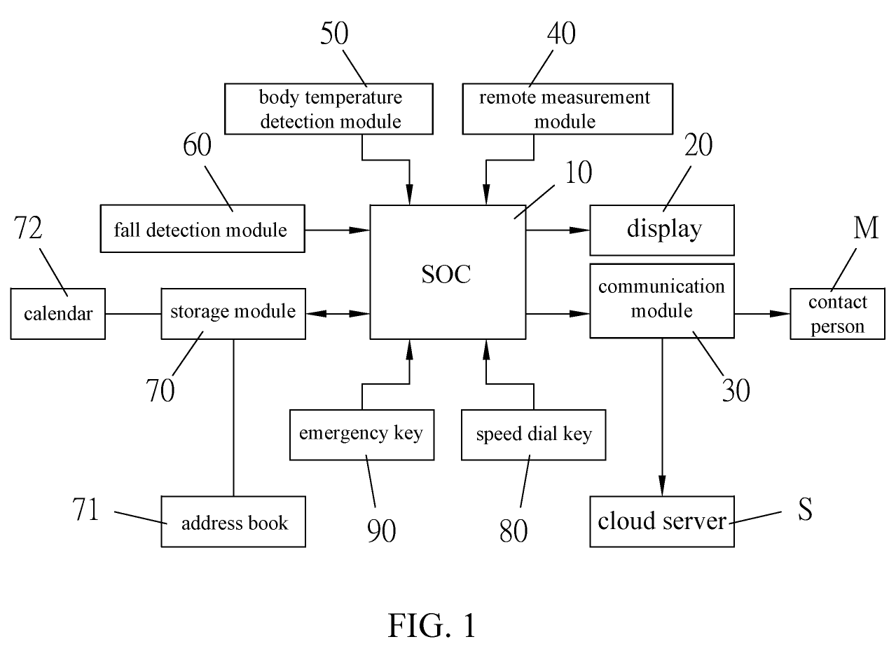

In order to facilitate the description of the contents of the present invention and the effects achieved thereby, specific embodiments will be illustrated with reference to the attached drawings. Reference being now made to FIG. 1, a lens-type medical detection device according to the present invention comprises:
a system on a chip (SoC) 10, which is connected to a display 20, a communication module 30, a remote measurement module 40, a body temperature detection module 50, and a fall detection module 60.

The communication module 30 is connected with a cloud server S and at least one contact person M. The cloud server S includes an artificial intelligence model S1.

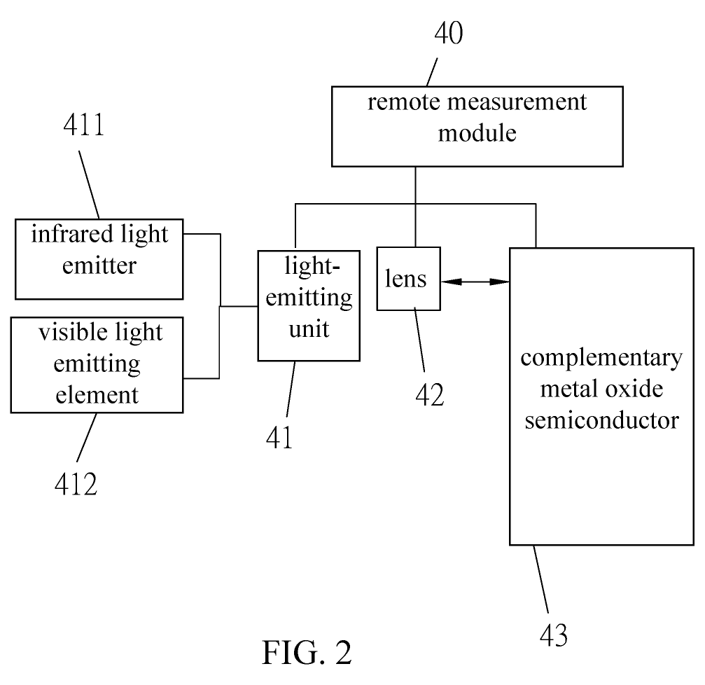

Referring to FIGS. 1 and 2, the remote measurement module 40 comprises at least one light-emitting unit 41 and at least one lens 42. The light-emitting unit 41 comprises at least one infrared light-emitting diode 411 and at least one visible light emitting element 412, wherein the remote measurement module 40 further comprises a complementary metal oxide semiconductor 43 (which is a CMOS photosensitive element) coupled with the lens 42.

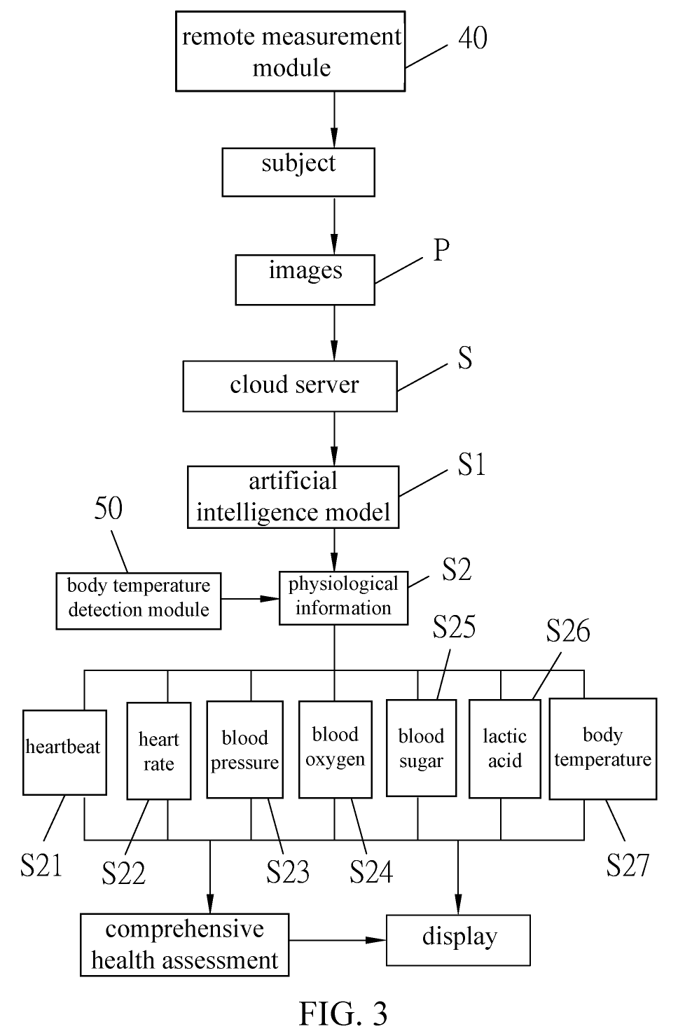

Referring to FIGS. 1-3, the light-emitting unit 41 is used to illuminate a subject, and then, the lens 42 is used to successively capture a plurality of images P and each of the images P is transmitted to the cloud server S to be subjected to computation performed by the artificial intelligence model S1 to generate physiological information S2, and the physiological information S2 is transmitted back and outputted to the display 20. The physiological information includes heartbeat S21, heart rate S22, blood pressure S23, blood oxygen S24, blood sugar S25, and lactic acid S26. Meanwhile, the artificial intelligence model S1 generates a comprehensive health assessment S3 according to the physiological information S2, so that the comprehensive health assessment S3 prompts the subject and his/her family to do proper handling corresponding thereto, such as going to the hospital for consultation, changing diet, and taking additional health supplements.

The artificial intelligence model S1 uses remote photoplethysmography (rPPG) and face recognition technology to process each of the images P to obtain the physiological information S2.

Remote photoplethysmography (rPPG) compares light reflected from the facial skin surface with light absorbed and scattered by the facial subcutaneous tissue, where the latter varies in response to the flow of blood in the capillaries. By means of continuously analyzing images within a short period of time for processing through the artificial intelligence model S1, multiple health data of the subject can be calculated. Remote photoplethysmography is used in conjunction with a facial recognition system and a machine learning algorithm to carry out signal processing on a signal of imaging photoplethysmogram, and a facial vascular signal is scanned through the lens 42, and then, the facial ROI signal to infer the relevant parameters of heart disease, heart rate and even blood pressure model. In the instant embodiment, each of the images P includes the face, chest, arms and wrists of the subject.

The body temperature detector 50 is provided for detection of the body temperature of the subject and integrates the body temperature S27 so measured into the physiological information S2.

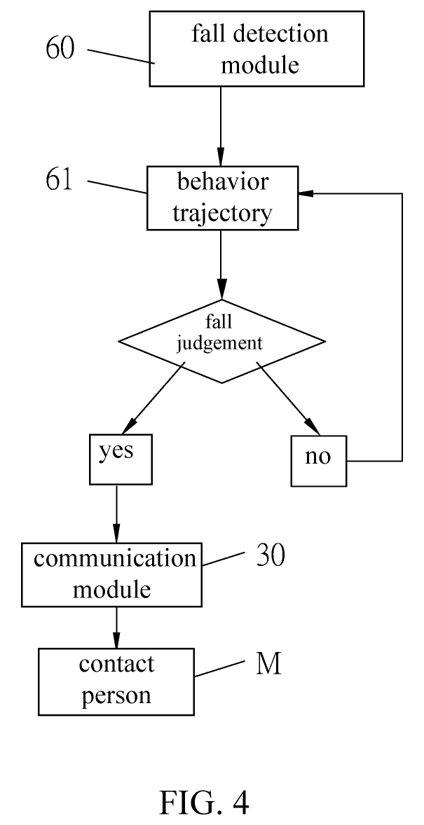

Referring to FIGS. 1 and 4, the fall detection module 60 receives a behavior trajectory 61 in real time in order to carry out fall judgement using the algorithm of the SoC 10 and to notify the contact person M through the communication module 30 when a fall is judged.

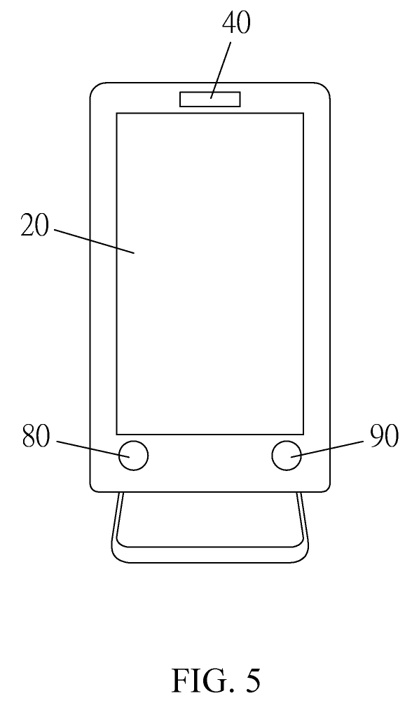

Referring to FIGS. 1 and 5, further, the SoC 10 is also connected to a storage module 70, and the storage module 70 includes an address book 71 for storing the information of the contact person M.

The SoC is connected to at least one speed dial key 80, and the speed dial key 80 is connected to the information of the at least one contact person M in the address book 71. The SoC is connected to at least one emergency key 90 and a loud speaker, and triggering the emergency key makes the loud speaker emit a warning sound effect.

The storage module includes a calendar 72 therein for recording and remining the subject to take medicine or go to the hospital for consultation on time.

I claim:

1. A lens-type medical detection device, comprising:
a system on a chip (SoC);
a display, which is electrically connected to the SoC;
a communication module, which is electrically connected to the SoC, wherein the communication module establishes connection with a cloud server and at least one contact person, the cloud server comprising an artificial intelligence model;
at least one remote measurement module, which is electrically connected to the SoC and comprises at least one light-emitting unit and at least one lens, the light-emitting unit comprising at least one infrared light-emitting diode and at least one visible light emitting element, wherein the light-emitting unit is operable to illuminate a subject and the lens is operable to successively capture a plurality of images and each of the images is transmitted to the cloud server to be subjected to computation performed by the artificial intelligence model to generate physiological information, the physiological information being transmitted back and outputted to the display, wherein the physiological information includes heartbeat, heart rate, blood pressure, blood oxygen, blood sugar, and lactic acid, and the artificial intelligence model generates a comprehensive health assessment according to the physiological information, so that the comprehensive health assessment reminds the subject and family thereof to carry out corresponding handling; and
a fall detection module, which is electrically connected to the SoC, wherein the fall detection module receives a behavior trajectory in real time in order to carry out fall judgement using an algorithm of the SoC and to notify the contact person through the communication module.

2. The lens-type medical detection device according to claim 1, wherein the remote measurement module further comprises a complementary metal oxide semiconductor.

3. The lens-type medical detection device according to claim 1, wherein the SoC is further connected to a body temperature detector, and the body temperature detector provides detection of a body temperature of the subject for integration into the physiological information.

4. The lens-type medical detection device according to claim 3, wherein the SoC is further connected to a storage module, and the storage module includes an address book for storing information of the contact person.

5. The lens-type medical detection device according to claim 4, wherein the SoC is connected to at least one speed dial key, and the speed dial key is connected with the information of the at least one contact person in the address book.

6. The lens-type medical detection device according to claim 5, wherein the SoC is connected to at least one emergency key and a loud speaker, and triggering of the emergency key makes the loud speaker emit a warning sound effect.

7. The lens-type medical detection device according to claim 4, wherein the storage module includes a calendar therein for recording and remining the subject to take medicine or go to the hospital for consultation.

8. The lens-type medical detection device according to claim 1, wherein the artificial intelligence model uses remote photoplethysmography (rPPG) and face recognition technology to process each of the images, in order to obtain the physiological information.

9. The lens-type medical detection device according to claim 8, wherein each of the images includes the face, chest, arms and wrists of the subject.
